(19) United States
(12) Patent Application Publication
(10) Pub. No.: US 2014/0377355 A1
(43) Pub. Date: Dec. 25, 2014

(54) Levofloxacin Inhalation Composition

(71) Applicant: Daniel Banov, Sugar Land, TX (US)

(72) Inventor: Daniel Banov, Sugar Land, TX (US)

(73) Assignee: PROFESSIONAL COMPOUNDING CENTERS OF AMERICA, Houston, TX (US)

(21) Appl. No.: 13/921,690

(22) Filed: Jun. 19, 2013

(51) Int. Cl. A61K 47/10; A61K 9/00; A61K 31/5383

(52) U.S. Cl. CPC A61K 47/10; A61K 31/5383; A61K 9/0073
USPC 424/489; 514/230.2

(57) ABSTRACT

An antibiotic inhalation composition for the treatment of bacteria related diseases in the respiratory tract is provided. The antibiotic inhalation composition may include a mixture of levofloxacin and a micronized poloxamer composition (excipient/solubilizer). Micronized poloxamer composition may include poloxamer 188 and poloxamer 407. The manufacturing method for micronized poloxamer composition may include any suitable process, such as non-contact mixing technology. This technology may include an apparatus for applying low-frequency acoustic field, in order to facilitate the mixing process. Antibiotic inhalation composition may be delivered to the respiratory tract employing any suitable inhalation devices, such as metered-dose inhalers (MDIs), dry powder inhalers, aerosols, syringe, pipette, forceps, measured spoon, eyedropper, nebulizers, or any suitable medically approved delivery apparatus. Furthermore, the synergistic effect of micronized poloxamer composition may provide improved solubility, dispersibility, and bioavailability of any suitable API within the antibiotic inhalation composition; thus, decreasing side effects and time of treatment.

### CROSS-REFERENCE TO RELATED APPLICATIONS

This application is related to U.S. Utility application Ser. No. 13/xxx,xxx, entitled Inhalation Composition for Treating Respiratory Tract Infections, and U.S. Utility application Ser. No. 13/xxx,xxx, entitled Poloxamer Based Inhalation Composition, filed on even date herewith.

### BACKGROUND

1. Field of the disclosure

The present disclosure relates in general to therapeutic formulations, and more particularly, to an antibiotic inhalation composition having levofloxacin.

2. Background Information

Antibiotics are substances used for stopping and treating infections from harmful microorganisms. Antibiotics are used in different forms, such as ointments, creams, gels, pills, sprays, or administrated directly into the body by absorption into the bloodstream. The administration method of an antibiotic usually determines how effective the treatment can be, however, it may also determine how severe the side effects may be.

The administration of a drug by inhalation is called a local treatment effected by a direct application of the drug to the affected area and may be expected to produce fewer side effects as compared with the general administration of a drug. However, the application of a drug by inhalation to the respiratory apparatus inclusive of naris, throat, trachea, and lung, may sometimes result in insufficient absorption of the drug through the mucous membrane depending upon the drug. Therefore, inhalation treatments are at a drawback in being unable to achieve enough indirect remedial effect attributable to an increase of the concentration of the drug in the blood. Additionally, it is impractical to administer some drugs by inhalation, as they irritate the mucous membrane, for instance of the respiratory tracts of the bronchi, causing coughing.

For the foregoing reasons, there is a need for drugs with increased absorption through the mucous membranes of the respiratory apparatus, improved dispersibility to the peripheral airways and alveoli, and which may include reduced side effects.

### SUMMARY

The present disclosure may include a therapeutic formulation for the treatment of bacterial infections in the respiratory tract. The formulation may be employed as an antibiotic inhalation composition. A method for preparing such composition is also described here.

The disclosed antibiotic composition may include at least one antibiotic agent as active pharmaceutical ingredient (API); additionally, antibiotic composition may include a combination of two or more poloxamers as excipients. According to an embodiment, a suitable API may be levofloxacin, while suitable poloxamers may include poloxamer 188 and poloxamer 407. Antibiotic inhalation composition may include poloxamer 188 in amounts of about 0.1% by weight to about 5.0% by weight, with 1.0% by weight being preferred, and poloxamer 407 in amounts of about 0.1% by weight to about 5.0% by weight, with 1.0% by weight being preferred.

Furthermore, the synergistic effect of micronized poloxamer composition may provide improved solubility and bioavailability of any suitable API. According to an embodiment, the manufacturing method for micronized poloxamer composition may include any suitable process, such as non-contact mixing technology. This technology may include an apparatus for applying low-frequency acoustic field, in order to facilitate the mixing process. Furthermore, this approach may allow creating micro-mixing zones through an entire mixing vessel, and therefore, it may allow providing a faster, more uniform mixing throughout a vessel.

In an embodiment, micronized poloxamer composition may include a particle size ranging between about 30 μm and about 70 μm, where 50 μm are preferred. The antibiotic inhalation composition may be obtained in powder form and may be used to fill capsules, which may be later employed for inhalation.

In other embodiments, antibiotic inhalation composition in powder form may be dissolved employing suitable solvents, such as sterile solution of sodium chloride and water, to obtain antibiotic composition in solution form. Antibiotic inhalation composition in solution form may be delivered to the respiratory tract using suitable inhalation devices, such as metered-dose inhalers (MDIs), dry powder inhalers, aerosols, syringe, pipette, measured spoon, eyedropper, nebulizers, or any suitable medically approved delivery apparatus.

Antibiotic inhalation composition may provide improved solubility and bio-availability of levofloxacin, thus decreasing treatment time and side effects occurrence. Antibiotic inhalation composition may be used for treating bacterial respiratory tract infections caused by bacteria, such as Bordetella pertussis, Streptococcus pneumoniae, Mycoplasma pneumoniae, Legionella pneumophila, and Chlamydia psittaci, among others.

Numerous other aspects, features and benefits of the present disclosure may be made apparent from the following detailed description taken together with the drawing figures.

### BRIEF DESCRIPTION OF THE DRAWINGS

The present disclosure can be better understood by referring to the following figures. The components in the figures are not necessarily to scale, emphasis instead being placed upon illustrating the principles of the disclosure. In the figures, reference numerals designate corresponding parts throughout the different views.

FIG. 1 is a micronized poloxamer composition block diagram, according to an embodiment.

FIG. 2 is an APIs block diagram in combination with micronized poloxamer composition, according to an embodiment.

### DETAILED DESCRIPTION

The present disclosure is here described in detail with reference to embodiments illustrated in the drawings, which form a part here. Other embodiments may be used and/or other changes may be made without departing from the spirit or scope of the present disclosure. The illustrative embodiments described in the detailed description are not meant to be limiting of the subject matter presented here.

### Definitions

As used here, the following terms may have the following definitions:

“Antibiotic” refers to an agent that destroys or inhibits bacterial growth.

“Excipient” refers to a substance added to a therapeutic formulation in order to provide suitable consistency or form the formulation.

“Poloxamer” refers to a non-ionic triblock copolymer having surfactant properties. Poloxamers may be used as thickening agents, gel formers, co-emulsifiers, solubilizers, and consistency enhancers in pharmaceutical compositions.

“Microprilling” refers to a process where solid spherical microprills may be produced from a liquid, tablets, or encapsulated ingredients with a diameter of few microns.

### Description

The present disclosure may relate to a pharmaceutical composition that, in one embodiment may be an antibiotic inhalation composition. The antibiotic inhalation composition may include a combination of two or more poloxamers as excipients and levofloxacin as an active pharmaceutical composition (API). According to an embodiment, disclosed antibiotic composition may be employed as an inhalation formulation for the treatment of bacterial infections in the respiratory tract.

Poloxamer Composition

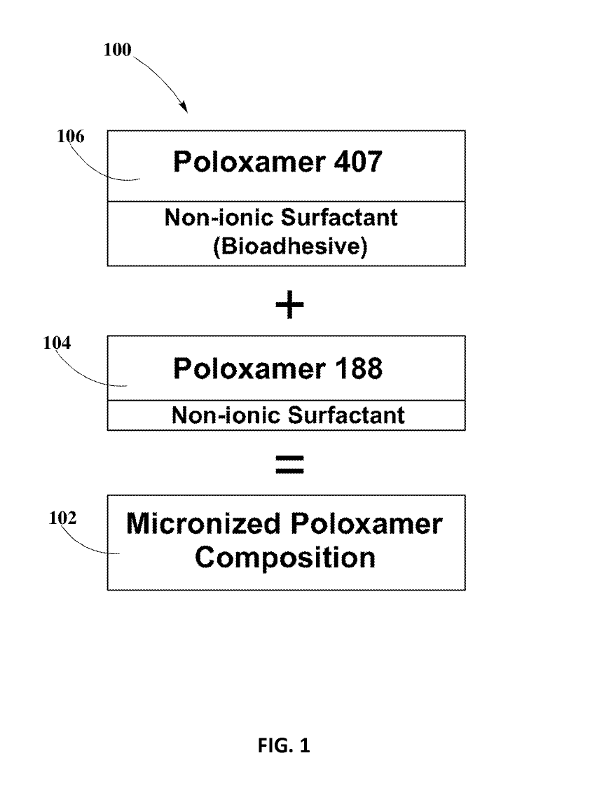

FIG. 1 is micronized poloxamer composition block diagram 100. The present disclosure may refer to an antibiotic inhalation composition used for treating bacterial infections in the respiratory tract. The antibiotic inhalation composition may include micronized poloxamer composition 102 as excipients. According to an embodiment, micronized poloxamer composition 102 may include poloxamer 188 104 in amounts of about 0.1% by weight to about 5.0% by weight, with about 1.0% by weight being preferred, and poloxamer 407 106 in amounts of about 0.1% by weight to about 5.0% by weight, with about 1.0% by weight being preferred.

The benefits of the microprilling process in poloxamer 188 104 and poloxamer 407 106 may include stronger solubilization properties, controlled dissolution rate, reduction of die-wall friction, achievement of homogeneous blend, elimination of dose dumping, and effectiveness as a water soluble lubricant. Micronized poloxamer composition 102 may include surfactant properties, where micronized poloxamer composition 102 may reduce the surface tension or the tension at the interface between any suitable solvent, such as water, and components, such as active pharmaceutical ingredients. Additionally, surfactant agents, such as micronized poloxamer composition 102, may include cleaning properties and may work as surface tension depressants, detergents, dispersing agents, and emulsifiers within any suitable composition, such as the disclosed antibiotic inhalation composition.

Furthermore, antibiotic inhalation composition may include solubility properties dictated by the hydrophobic portion of the poloxamers. The use of micronized poloxamer composition 102 may increase the solubility of the active pharmaceutical ingredient (API) that is employed, thus the drug may include enhanced treatment properties. Furthermore, the properties of each poloxamer may vary in terms of molecular weight, appearance, hydrophilicity/hydrophobicity, and solubility, which may be determined by the chain length of the polyxyethylene (EO-) units and polyoxypropyene (PO-) units.

According to an embodiment, micronized poloxamer composition 102 in combination with a suitable API, may decrease the minimum inhibitory concentration (MIC) for microorganisms such as Escherichia coli, Staphylococcus aureus, Pseudomonas aeruginosa, Candida albicans, Aspergillus niger, Salmonella typhimurium, methicillin resistant Staphylococcus aureus, Aspergillus fumigatus, and Rhizopus oryzae, among others. This may be achieved by allowing a more uniform dispersion as a result of the narrow distribution of particles from an API.

Manufacturing Method of Micronized Poloxamer Composition

The manufacturing method for micronized poloxamer composition 102 may include a non-contact mixing technology. This technology may include an apparatus for applying low-frequency acoustic field, in order to facilitate the mixing process. Furthermore, this approach may allow creating micro-mixing zones through an entire mixing vessel, and therefore, it may allow providing a faster, more uniform mixing throughout a vessel; thus, decreasing side effects and time of treatment.

According to an embodiment, micronized poloxamer composition 102 may be obtained in powder form having a particle size between about 30 μm and about 70 μm, with about 50 μm being preferred. Micronized poloxamer composition 102 in a powder form may be employed to fill capsules, which may be used for inhalation.

Antibiotic Inhalation Composition

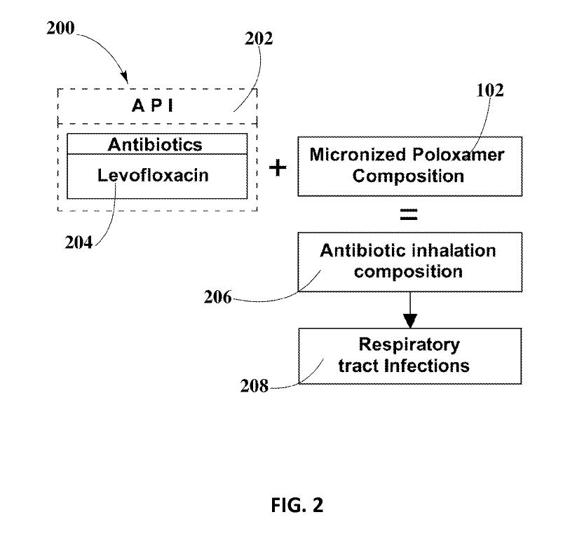

FIG. 2 is an antibiotic inhalation composition block diagram 200, where APIs 202 may be in combination with micronized poloxamer composition 102 to form antibiotic inhalation composition 206, according to an embodiment. Specifically, micronized poloxamer composition 102 may be used in combination with any suitable API 202, such as levofloxacin 204, for treating bacterial respiratory tract infections 208. According to an embodiment, levofloxacin 204 may be mixed with micronized poloxamer composition 102, which may be previously dissolved in a suitable solvent, in order to produce antibiotic inhalation composition 206. Suitable solvents may be water, saline solution, or sodium chloride solution, among others.

According to an embodiment, levofloxacin 204 may be administered in dosage of about 50 ml to about 150 ml. Additionally, the antibiotic composition may be administered intranasally or by inhalation, among others. According to some embodiments, micronized poloxamer composition 102 in combination with suitable levofloxacin 204 may be used for treating bacterial respiratory tract infections 208, such as Haemophilus influenzae, Klebsiella pneumoniae, Methicillin-sensitive but not methicillin resistant Staphylococcus aureus, Streptococcus pneumoniae, Chlamydophila pneumoniae, and Mycoplasma Pneumonia, among others.

In an embodiment, antibiotic inhalation composition 206 may be delivered to the respiratory tract employing suitable devices, such as metered-dose inhalers (MDIs), dry powder inhalers, intranasal sprays, aerosols, syringe, pipette, forceps, measured spoon, eyedropper, nebulizers, drops, or any suitable medically approved delivery apparatus. Specifically, micronized poloxamer composition 102 may be delivered directly to the respiratory tract via nasal aerosol sprays. The administration of the aerosol may vary according to subject's age, weight, and the severity and response of the symptoms.

In a further embodiment, micronized poloxamer composition 102 may increase the solubility and action of APIs 202 ingredients, specifically, levofloxacin 204. Furthermore, the synergistic effect of micronized poloxamer composition 102 may provide an improved solubility, dispersibility, and bioavailability of any suitable API 202 within antibiotic inhalation composition 206.

Antibiotic inhalation composition 206 in solution form may include between about 2 ml to about 10 ml of solvent, and about 5 mg to about 5 g of antibiotic inhalation composition 206, where about 1 g to about 2 g may be preferred. According to another embodiment, antibiotic inhalation composition 206 may be delivered in humans in amounts of about 2 ml to about 10 ml, where about 5 ml may be preferred.

In other embodiments, antibiotic inhalation composition 206 in powder form may be dissolved in order to obtain a solution form. Suitable solvents may include sterile solution of sodium chloride and water, among others. Additionally, antibiotic inhalation composition 206 may reduce levofloxacin 204 side effects, such as chest pain and severe dizziness, fainting, fast or pounding heartbeats, severe headache, ringing in the ears, nausea, vision problems, and pain behind the eyes, among others.

Levofloxacin

Levofloxacin 204 is an antibiotic of the fluoroquinolone drug class. The spectrum of activity for this drug includes several bacterial pathogens (e.g. Escherichia coli, Haemophilus influenzae, Klebsiella pneumoniae, Legionella pneumophila, Moraxella catarrhalis, Proteus mirabilis, Pseudomonas aeruginosa, Staphylococcus aureus, Streptococcus pneumoniae, Staphylococcus epidermidis, Enterococcus faecalis, and Streptococcus pyogenes).

Levofloxacin 204 may be used to treat infections, such as pneumonia, chronic bronchitis and sinues, urinary tract, kidney, prostate, and skin infections, among others. Levofloxacin 204 may also be used to treat people who have been exposed to anthrax germs. Furthermore, levofloxacin 204 may also be used to treat endocarditis, sexually transmitted diseases, and tuberculosis (TB). Levofloxacin 204 is also used to prevent or treat traveler's diarrhea and plague.

### EXAMPLES

Example #1 is an embodiment of micronized poloxamer composition 102, where instead of employing poloxamer 188 104 and poloxamer 407 106 as excipients, other suitable poloxamers may be used. Suitable micronized poloxamer composition 102 may include: poloxamer 101, poloxamer 105, poloxamer 108, poloxamer 122, poloxamer 124, poloxamer 181, poloxamer 182, poloxamer 183, poloxamer 184, poloxamer 185, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, and combinations thereof.

Example #2 is an embodiment of antibiotic inhalation composition 206, where micronized poloxamer composition 102 may be used in combination with xylitol or sugar alcohol. Xylitol may be included in amounts of about 50% by weight to about 90% by weight, most suitable being 80% by weight.

Example #3 is an application of micronized poloxamer composition 102 in combination with any suitable API 202, such as levofloxacin 204, which may be used for treating bacterial respiratory tract infections 208 in animals, applying suitable dosages according to the weight and size of the animal.

While various aspects and embodiments have been disclosed here, other aspects and embodiments may be contemplated. The various aspects and embodiments disclosed here are for purposes of illustration and are not intended to be limiting, with the true scope and spirit being indicated by the following claims.

What is claimed is:

1. A composition for prevention and treatment of infections of the respiratory tract caused by bacteria, comprising levofloxacin and at least two poloxamers.

2. The composition according to claim 1, wherein one of the at least two poloxamers is selected from the group consisting of poloxamer 188, poloxamer 407, and combinations thereof.

3. The composition according to claim 1, wherein one of the at least two poloxamers is poloxamer 188.

4. The composition according to claim 3, wherein the poloxamer 188 is about 0.1% by weight to about 5% by weight.

5. The composition according to claim 3, wherein the poloxamer 188 is about 1% by weight.

6. The composition according to claim 1, wherein one of the at least two poloxamers is poloxamer 407.

7. The composition according to claim 6, wherein the poloxamer 407 is about 0.1% by weight to about 5% by weight.

8. The composition according to claim 6, wherein the poloxamer 407 is about 1% by weight.

9. The composition according to claim 1, wherein one of the at least two poloxamers is micronized.

10. The composition according to claim 1, wherein one of the at least two poloxamers comprises a particle size of about 30 μm to about 70 μm.

11. The composition according to claim 1, wherein one of the at least two poloxamers comprises a particle size of about 50 μm.

12. A method for prevention and treatment of infections of the respiratory tract caused by bacteria, comprising administering to a patient in need of such treatment a formulation comprising levofloxacin and at least two poloxamers.

13. The method according to claim 12, wherein one of the at least two poloxamers is selected from the group consisting of poloxamer 188, poloxamer 407, and combinations thereof.

14. The method according to claim 12, wherein one of the at least two poloxamers is poloxamer 188.

15. The method according to claim 14, wherein the poloxamer 188 is about 0.1% by weight to about 5% by weight.

16. The method according to claim 14, wherein the poloxamer 188 is about 1% by weight.

17. The method according to claim 12, wherein one of the at least two poloxamers is poloxamer 407.

18. The method according to claim 17, wherein the poloxamer 407 is about 0.1% by weight to about 5% by weight.

19. The method according to claim 17, wherein the poloxamer 407 is about 1% by weight.

20. The method according to claim 12, wherein the formulation is a powder.

21. The method according to claim 20, wherein the powder is dissolved in a solvent comprising saline.

22. The method according to claim 12, wherein the formulation is administered using an inhalation device selected from the group consisting of a metered-dose inhalers (MDIs), a dry powder inhalers, and a nebulizer.

22. The method according to claim 12, wherein the formulation is administered using one selected from the group consisting of a syringe, pipette, measured spoon, and eyedropper.

23. The method according to claim 12, wherein the infections of the respiratory tract are selected from the group consisting of Bordetella pertussis, Streptococcus pneumoniae, Mycoplasma pneumoniae, Legionella pneumophila, and Chlamydia psittaci.
